(19) United States
(12) Patent Application Publication
(10) Pub. No.: US 2017/0042532 A1
(43) Pub. Date: Feb. 16, 2017

(54) SUTURE CUTTER

(71) Applicant: TECHNICAL MANUFACTURING WEST, LLC, VALENCIA, CA (US)

(72) Inventors: JOHN JAMES VALADEZ, Santa Clarita, CA (US); KEVIN GLEN, Ventura, CA (US); TODD RUSH, Santa Clarita, CA (US)

(21) Appl. No.: 15/234,421

(22) Filed: Aug. 11, 2016

Related U.S. Application Data
(60) Provisional application No. 62/204,308, filed on Aug. 12, 2015.

(51) Int. Cl. A61B 17/04

(52) U.S. Cl. CPC A61B 17/0467; A61B 17/0482; A61B 2017/00367

(57) ABSTRACT

A suture cutter device is disclosed. Embodiments provide a cutting tip capable of cutting higher strength sutures such as UHMWPE based sutures. A shaft includes a cutting tip with a low profile capable of use in minimally invasive procedures with tiny incisions. The tip may include for example a slot, nook, or tunnel to hold a suture. A sleeve housing the shaft may include a blade edge aligned with the suture. Actuation of the device draws the suture toward the blade edge for cutting.

### CROSS-REFERENCE TO RELATED APPLICATION

This application claims benefit under 35 U.S.C. §119(e) of U.S. Provisional Application having Ser. No. 62/204308 filed Aug. 12, 2015, which is hereby incorporated by reference herein in its entirety.

### FIELD OF THE INVENTION

The present invention relates in general to medical devices. More particularly, the invention is directed to a suture cutter.

### BACKGROUND OF THE INVENTION

During surgery, suture is often used as a means to keep tissue approximated to implants or tissues against other tissues. This is normally done to minimize movement so the body can heal properly in the immediate area.

After tying a knot to fixate the suture in place, the excess suture limbs need to be cut and removed. In an open procedure or on the skin surface this can be done with a pair of scissors or similar instruments. With the advent of minimally invasive surgery where much of the work is done through a small opening such as a cannula, a standard surgical scissor is too big to fit through the opening to cut the suture.

Some devices have been developed that have the ability to cut a suture with a mechanism that is placed on the end of a long rod that can fit in to these tight spaces. This has worked well with most sutures until stronger sutures such as those made with Ultra-high-molecular-weight polyethylene (UHMWPE) came on to market. Due to their strength, these sutures proved difficult to cut. This forced designers to come up with more robust systems for cutting this suture of which there are a few on the market.

The problem is that current devices do not cleanly cut all the fibers of the suture braid. They also have limits on the amount of or number of times they can be used to cut a suture during the instrument's lifetime.

Accordingly, a need exists to provide an inexpensive suture cutter that will have a long life of cleanly cutting sutures.

### SUMMARY OF THE INVENTION

In a first aspect, a suture cutter device is disclosed. The suture cutter includes a housing and a handle coupled to the housing. A shaft may be coupled to the handle. A tip of the shaft may be positioned distally from the handle. An opening in the tip is configured for receiving a suture. A sleeve may surround the shaft. The suture cutter may also include an actuation mechanism for pushing the sleeve over the shaft or retracting the shaft into the sleeve. A blade edge may be positioned on a distal end of the sleeve and disposed to cut the suture in the opening of the tip in response to the actuation mechanism pushing the sleeve over the shaft or retracting the shaft into the sleeve.

In a second aspect, a suture cutter device is disclosed. The suture cutter includes a housing and a shaft coupled to the housing. A tip of the shaft may be positioned on a distal end of the housing. A blade edge may be positioned on an edge of the housing. An opening in the tip of the shaft configured for receiving a suture. The suture cutter device may also include an actuation mechanism coupled to the housing for moving the opening in the tip toward the blade edge, wherein the suture in the opening of the tip is cut by the blade edge in response to the actuation mechanism being operated.

In a third aspect, a suture cutter device is disclosed. The suture cutter includes a housing and a handle coupled to the housing. A shaft may be coupled to the handle. A tip of the shaft may be positioned distally from the handle. An opening in the tip is configured for receiving a suture. A sleeve may surround the shaft. The suture cutter may include a spring-loaded actuation mechanism for pushing the sleeve distally over the shaft or retracting the shaft into the sleeve. A first blade edge is positioned on a distal end of the sleeve and a second blade edge is positioned on the tip of the shaft in alignment with the first blade edge. The first blade edge and the second blade edge are disposed to cut the suture in the opening of the tip in response to the actuation mechanism pushing the sleeve over the shaft or retracting the shaft into the sleeve. The opening in the tip includes a slot and a nook in the slot disposed to position the suture in alignment with the blade edge.

These and other features and advantages of the invention will become more apparent with a description of preferred embodiments in reference to the associated drawings.

### DESCRIPTION OF THE DRAWINGS

FIG. 1 is a top perspective view of a suture cutter in an embodiment of the subject technology.

FIG. 2 is a bottom perspective view of the suture cutter of FIG. 1.

FIG. 3 is a partial cross-sectional side view of the suture cutter along line 3-3 of FIG. 1 showing an actuation mechanism without the cutting tip in an embodiment of the subject technology.

FIG. 3A is a side perspective view of a button assembly of FIG. 3 in an embodiment of the subject technology.

FIG. 3B is a perspective, partial cross-sectional view of the suture cutter shown in FIG. 3 without the cutting tip.

FIG. 4 is an enlarged left side perspective view of a cutting tip in an extended position according to an embodiment of the subject technology.

FIG. 4A is an enlarged rotated perspective view of the cutting tip of FIG. 4.

FIG. 4B is a cross-sectional view taken along the line 4B-4B of FIG. 4A.

FIG. 5 is an enlarged right side perspective view of the cutting tip of FIG. 4.

FIG. 6 is an enlarged perspective end view of the cutting tip of FIG. 4.

FIG. 7 is an enlarged left side perspective end view of a cutting tip in an alternate embodiment of the subject technology.

FIG. 8 is an enlarged right side perspective end view of the cutting tip of FIG. 7.

FIG. 9 is an enlarged perspective bottom view of the cutting tip of FIG. 7.

FIG. 10 is a partial cross-sectional side view of a suture cutter showing an alternate embodiment of an actuation mechanism without the cutting tip.

FIG. 11 is a side perspective view of a cutting tip according to another embodiment.

FIG. 12 is a perspective end view of the cutting tip of FIG. 11.

FIG. 13 is a side perspective view of the cutting tip of FIG. 11 in a retracted cutting position.

FIG. 14A is a cross-sectional view of an actuation mechanism for the cutting tip of FIGS. 11 and 14B in a default position.

FIG. 14B is a side view of the cutting tip of FIG. 11 in the default position of the actuation mechanism associated with FIG. 14A.

FIG. 15A is a cross-sectional view of the actuation mechanism of FIG. 14A in an actuated position.

FIG. 15B is a side view of the cutting tip of FIG. 14A in an actuated, cutting position associated with the actuated position of the actuation mechanism of FIG. 15A.

FIG. 16 is a front perspective view of a gate in the actuation mechanism of FIG. 14A according to another embodiment.

FIG. 17 is a front view of the gate of FIG. 16.

### DETAILED DESCRIPTION OF THE PREFERRED EMBODIMENTS

The following preferred embodiments, in general, are directed to a suture cutter device. As will be appreciated, aspects of the suture cutting device and its embodiments provide convenience for cutting the limbs of suture and particularly those suture limbs created in minimally invasive procedures. Moreover, aspects disclosed are useful and superior to conventional suture cutting devices because the preferred elements provide a reliable and strong cutting force that can cleanly cut UHMWPE based sutures. In general, preferred devices are disclosed which include a tip for holding a suture drawn to a blade edge by an actuation mechanism. It will be understood that the embodiments disclosed may include different tips and actuation mechanisms in combination and no one tip is necessarily operated with any particular actuation mechanism disclosed. Thus enumeration in the following does not imply that a tip with the same number series must be operated by an actuation mechanism of the same number series.

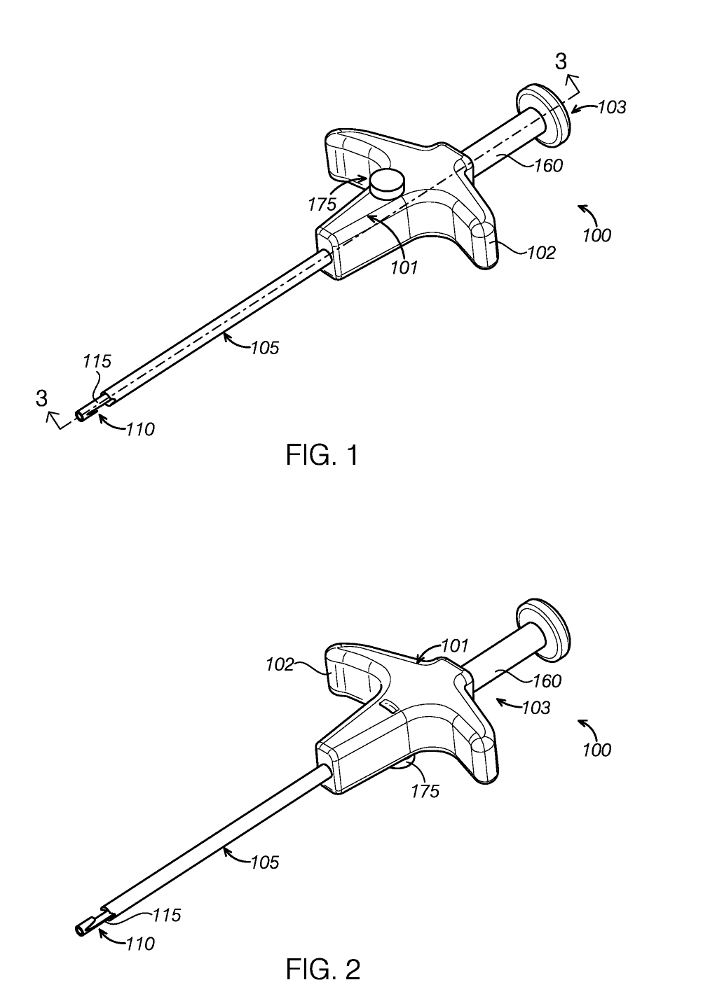

Referring now to FIGS. 1 and 2, a suture cutter device 100 (referred to generally as the “device 100” or simply as the “device”) is shown according to an exemplary embodiment. The device 100 includes in general a housing 101 housing a shaft 115 with a distal tip 110 configured to carry and cut a suture (not shown) and an actuation mechanism 103. In a general embodiment (and shown in more detail below), it will be appreciated that the tip 110 includes an opening for holding the suture and the housing 101 includes a cutting device proximate an edge of the housing that cuts the suture when the tip 110 is actuated toward the housing 101, such that the tip 110 is small enough to fit within a cannula and still be manipulated for suturing (with one or more sutures and/or suture tapes if desired). The sleeve 105 can also be attached to the plunger 160 such that actuation pushes the sleeve 105 distally over the tip 110 which is held stationary relative to the housing 101.

In an exemplary embodiment, the shaft 115 is housed co-axially (and/or circumferentially) by a sleeve 105. The tip 110 is integrated into a distal end of the shaft 115. The actuation mechanism 103 may include a handle 102 and a plunger 160 coupled axially and proximally to the shaft 115. The tip 110 is preferably positioned on an end distal to the plunger 160. It will be appreciated that the cutting tip 110 has a low profile that can be used for minimally invasive procedures yet incorporates features capable of providing knot pushing and cutting of high strength sutures. As an example and not by way of limitation, the profile of the cutting tip 110 may have the following preferred ranges of diameters/cross-dimensions: 1.5 to 4 mm

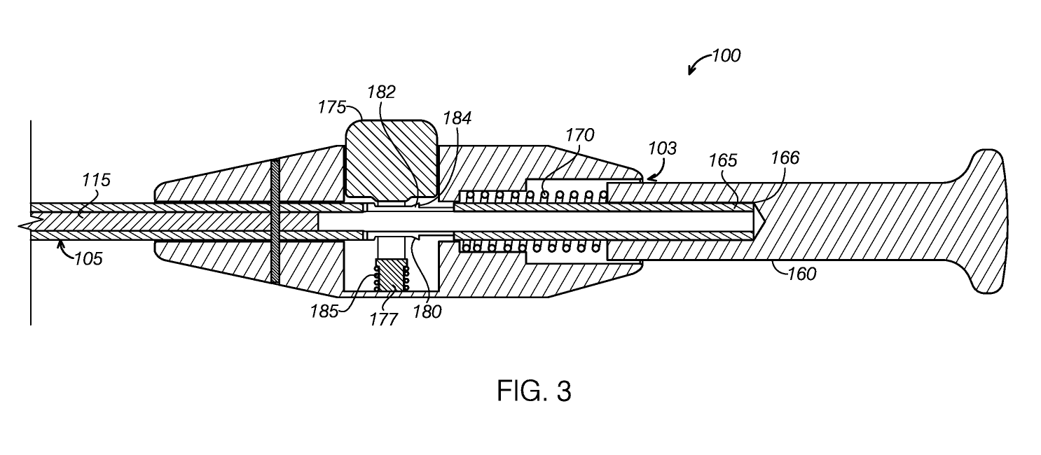

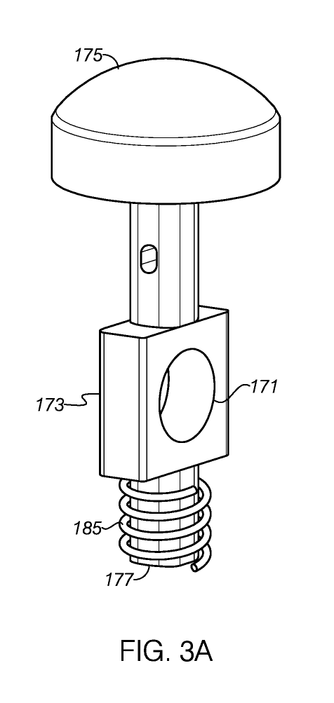

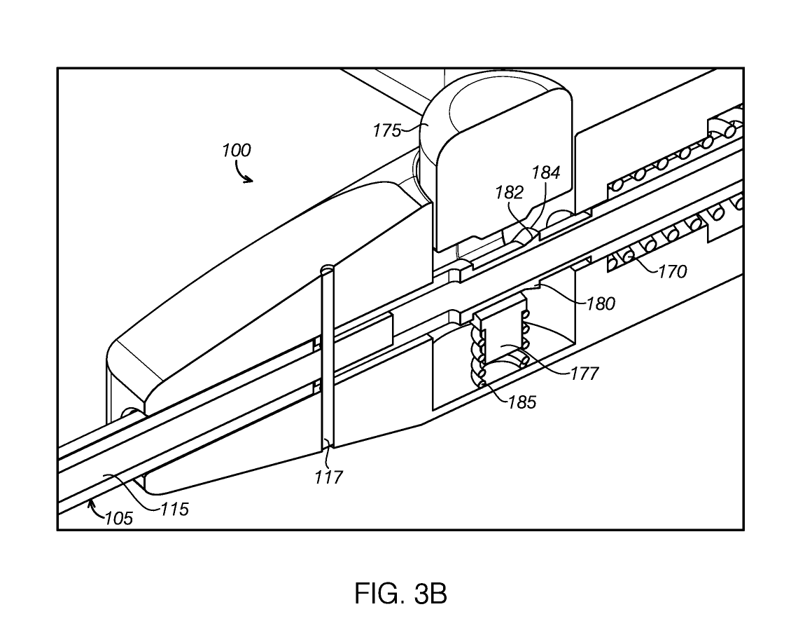

An exemplary embodiment of an actuation mechanism is shown in FIGS. 3 and 3A-3C. The device 100 may include a button 175 coupled to a connection between the shaft 115 and the plunger 160. For example, a connecting rod 165 may be attached to the plunger 160 on a proximal end 166 and the sleeve 105 may be the opposite distal end. In some embodiments, the sleeve 105 is integral with the rod 165 formed in a uni-body construction. The sleeve 105, within a distal end of the housing 101, may include a hollow section of length for receipt of the shaft 115. The shaft 115 may be secured into a fixed position relative to the housing 101 within the sleeve 105 by a cross pin 117. A button piston 177 may hold a spring 185 to bias activation of the button 175. The button piston 177 may include a gate 173, shown in FIG. 3A, with a transverse opening 171 for receipt of the rod 165 therethrough. The rod 165 may be hollow or solid. In some embodiments, the rod 165 may include a detent 180 abutting the piston 177 locking the rod 165 from moving axially. The detent 180 may include a sloped ramp 182 and trailing vertical wall 184. Activation of the button 175 allows the plunger 160 to move the rod 165 distally and axially, and the detent 180 through the piston opening. In response, the sleeve 105 moves distally and co-axially over the shaft 115 so that the distal edge of the sleeve 105 is drawn forward toward the tip 110. In embodiments with the detent 180, the rod 165 moves through the gate 173. The sloped ramp 182 abuts the inner circumference of the opening 171 until the sloped ramp 182 passes the width of the gate 173 and the trailing vertical wall 184 is on the inner face of the gate 173. The sloped ramp 182 allows the option of being able to advance the plunger 160 without actuating the button 175 until the gate 173 is locked within the detent 180.

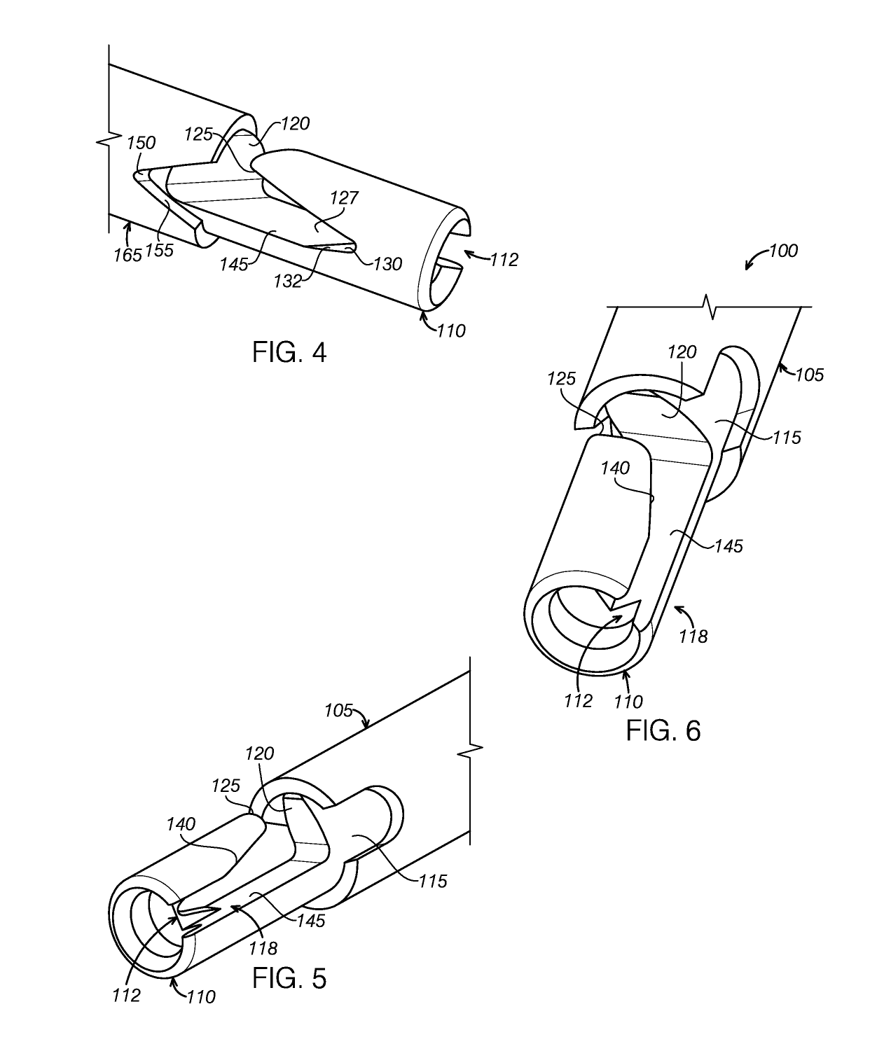

Referring now to FIGS. 4, 4A, 4B, and 5-6, an exemplary embodiment of the tip 110 is shown at a distal end of the sleeve 105. When the device 100 is in a default state, the tip 110 is generally projecting out farther than the distal end of the sleeve 105. In an exemplary embodiment, the sleeve 105 includes at its distal end a blade edge 150 against which the suture will be drawn to, to cut by the remaining features of the tip 110 when the device 100 is actuated. In some embodiments, the sleeve 105 may include a tapering guide wall 155 to help position the suture and draw it into an optimal cutting position. In some embodiments, the guide wall 155 may have a V-shape with the blade edge 150 being positioned at the nadir of the “V”.

The tip 110 may be open at its distal end to provide lacing a suture into securement. The tip 110 may include an opening 112 which includes an entrance channel 118 defined by a sidewall 140 and a horizontal platform 145. The opening 112 may allow the user to push knots into a desired position. The sidewall 140 may include a hairpin turn sidewall 125. The hairpin turn sidewall 125 leads to a receiving slot 127 (FIG. 4) which may include a distal nook 130 at a terminal point of the hairpin sidewall 125 for securing the suture as the actuation mechanism (FIG. 1) is operated. The nook 130 may include a blade edge 132. A wall 120 extending generally across the diameter of the shaft 11 may be positioned opposite the distal end of the tip 110 and connected to the platform 145. The wall 120 may help guide the suture around the hairpin sidewall 125 into the distally extending receiving slot 127. The nook 130 may be aligned along the same plane with the blade edge 150.

In operation, the user may feed the suture through the entrance channel 118 and around the hairpin sidewall 125 until the suture comes to rest within the nook 130. As the device 100 is actuated, sleeve 105 and blade edge 150 are drawn co-axially and distally over the tip 110 until the blade edge 150 makes contact with the suture positioned against the blade edge 132 in the nook 130. The user may pin the suture within the chamfered edges of the guide wall 155 and the nook 130 which form a closed diamond shaped pocket so the cutter can be slid along the suture but not lose engagement. If a cut is desired, the user continues with actuation so that the blade edge 150 continues to slide over and past the blade edge 132 and nook 130 providing a guillotine or scissor action to the suture with the two nook edges coming together. It will be appreciated that this guillotine/scissor action will be strong enough to cut through many suture materials including UHMWPE-based sutures, several constructs such as round and ribbon and more than one strand at a time. In some embodiments, the actuation mechanism 103 for example, includes stages of movement for the sleeve 105 to provide either the knot pushing position or the cutting position.

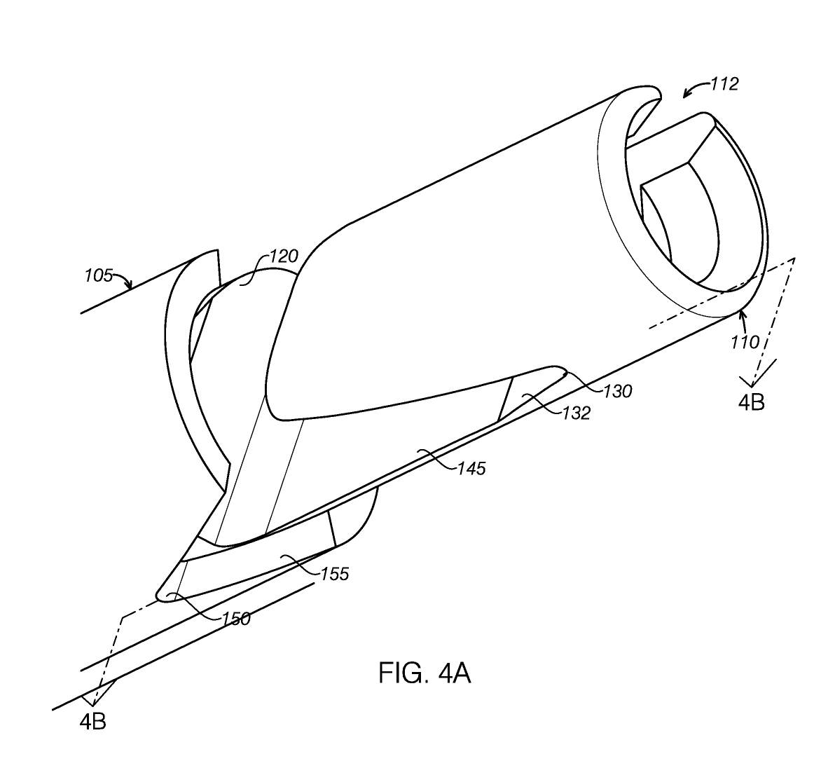

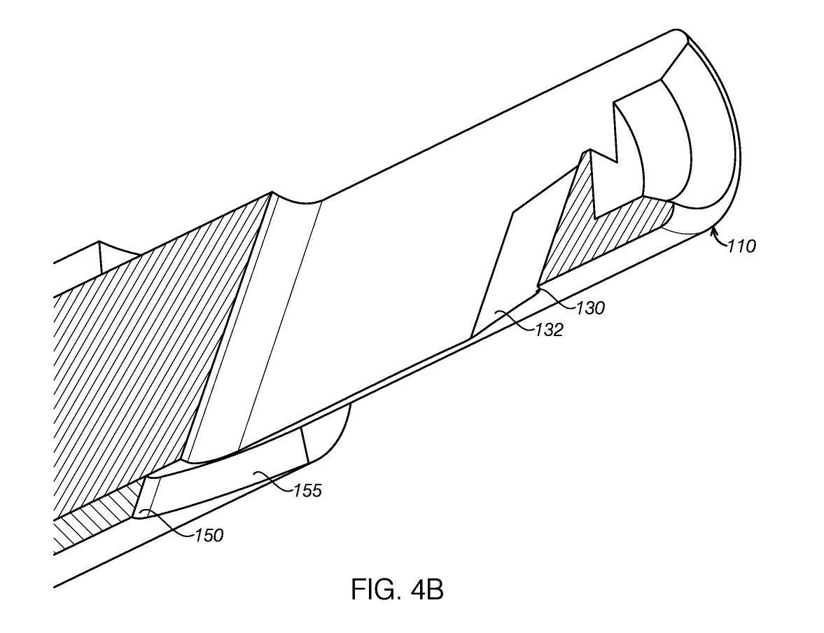

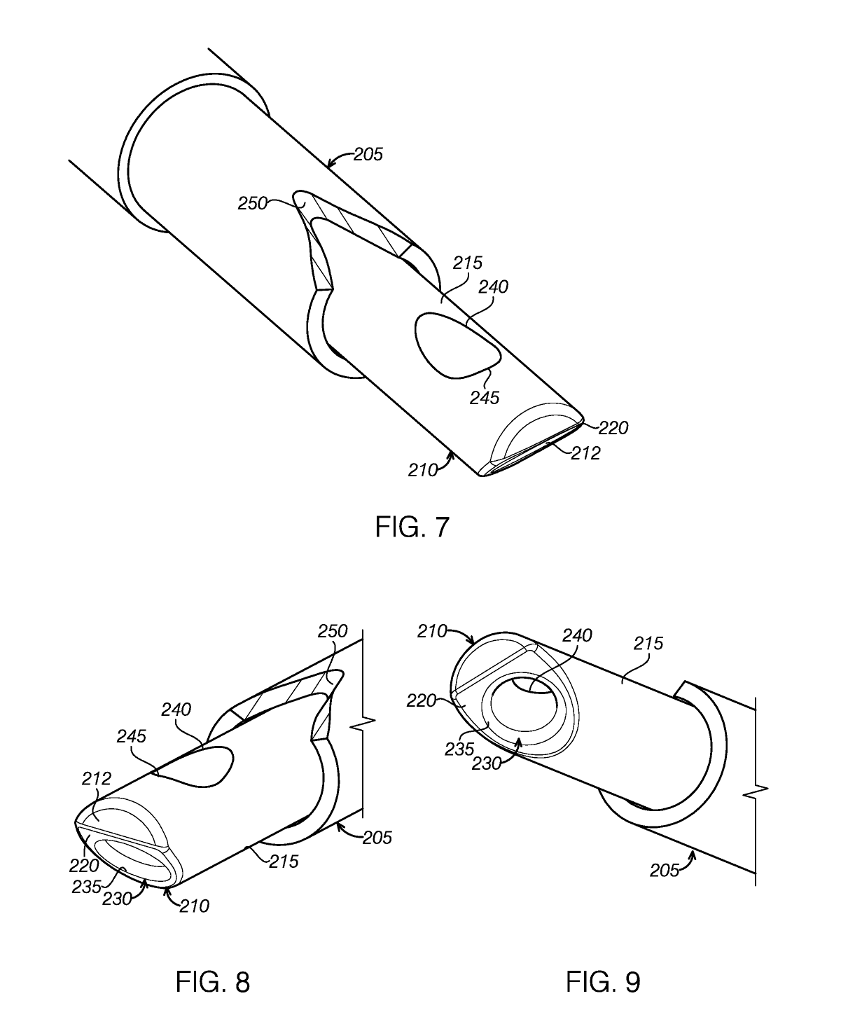

Referring now to FIGS. 7-9, an alternate embodiment of a cutting tip 210 of a shaft 215 is shown. The tip 210 is closed at its distal end 212. A sleeve 205 includes a blade edge 250 similar to the blade edge 150. The shaft 215 is similar to the shaft 115 of the embodiment discussed above. However, the tip 210 includes a transversely facing guide opening, or through hole, 230 disposed to position the suture in alignment with the blade edge 250. The guide opening 230 may be a pocket hole formed on a beveled end 220 extending from the end 230 at an angle from one side of the shaft 215 to an opposite side of the shaft 215 defining an exit 240. The exit 240 may include a blade edge 245. The blade edge 245 is aligned with the blade edge 250. In some embodiments, the guide opening 230 may include a chamfered entrance 235. In operation, the user threads the suture through the bottom of the guide opening 230. The chamfered entrance 235 may help guide the suture end into the pocket hole and out the exit 240. As the user actuates the device, the sleeve 205 is drawn toward the suture which is positioned between the blade edge 250 and a blade edge 245 of the exit 240 and cut the suture when the blade edge 250 moves past the blade edge 245.

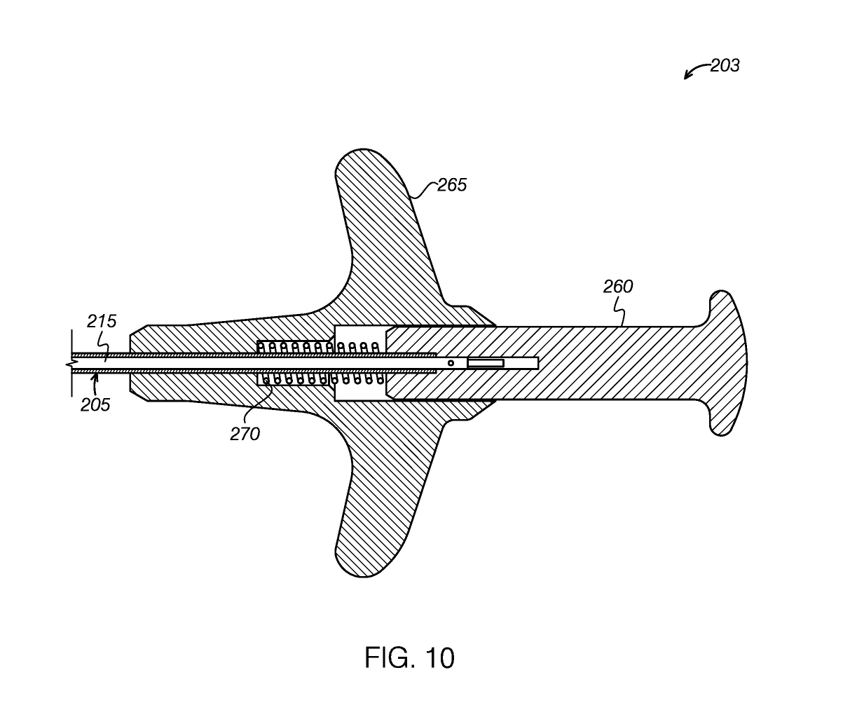

Referring now to FIG. 10, an alternate embodiment of an actuation mechanism 203 is shown. The actuation 203 is similar to the actuation 103 except that the mechanism 203 does not include a push button 175 to provide a locked position of the tip 210. In operation, the user grasps handle 265 and presses simultaneously against the plunger 260 which is biased by spring 270. Actuation of the plunger 260 pushes the sleeve 205 distally and co-axially over the shaft 215 held stationary relative to the handle 265 to move for example, the blade edge 250 distally over the cutting tip 210 as described with respect to FIGS. 7-9. After the cut, the spring 270 releases the plunger 260 and sleeve 205 proximally back to their default positions.

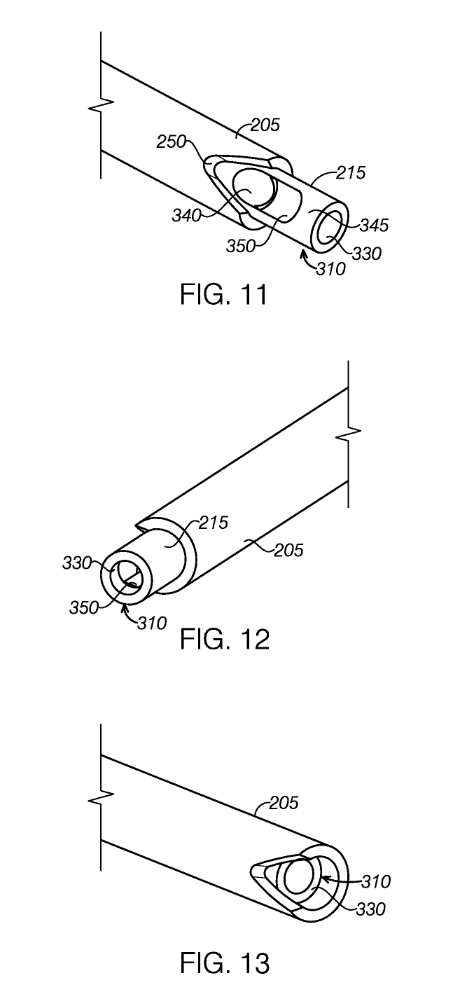

Referring now to FIGS. 11-13 and 14A, 14B, 15A, and 15B, an alternate embodiment of a suture cutting tip 310 is shown from different angles. The tip 310 operates similar to tip 210 except that the tip 310 includes a planar end face with a distal opening 330. The distal opening 330 is an entrance to a tunnel 350 with an exit 340 on a side wall of the shaft 215. The distal opening 330 may be chamfered enhancing the tip's ability to push a suture knot without fraying the suture. If the feature is in effect, this is the position of the safety lockout. The tunnel exit 340 is aligned with the blade edge 250. The tunnel 350 acts as a captured pathway to hold the suture in the tip 310 while allowing the user to slide the device along a suture strand in line with the tunnel's axis. The suture is normally placed through the distal opening 330 and out the tunnel exit 340 though the reverse configuration is contemplated. The wider and rounded slot provided by the tunnel 350 allows for freer suture movement. FIG. 13 shows the tip 310 in the cutting position. With the suture in the tunnel 350, the blade edge 250 can be slid over the tunnel exit 340 and blade edge 345 effectively acting as a guillotine to cut the suture.

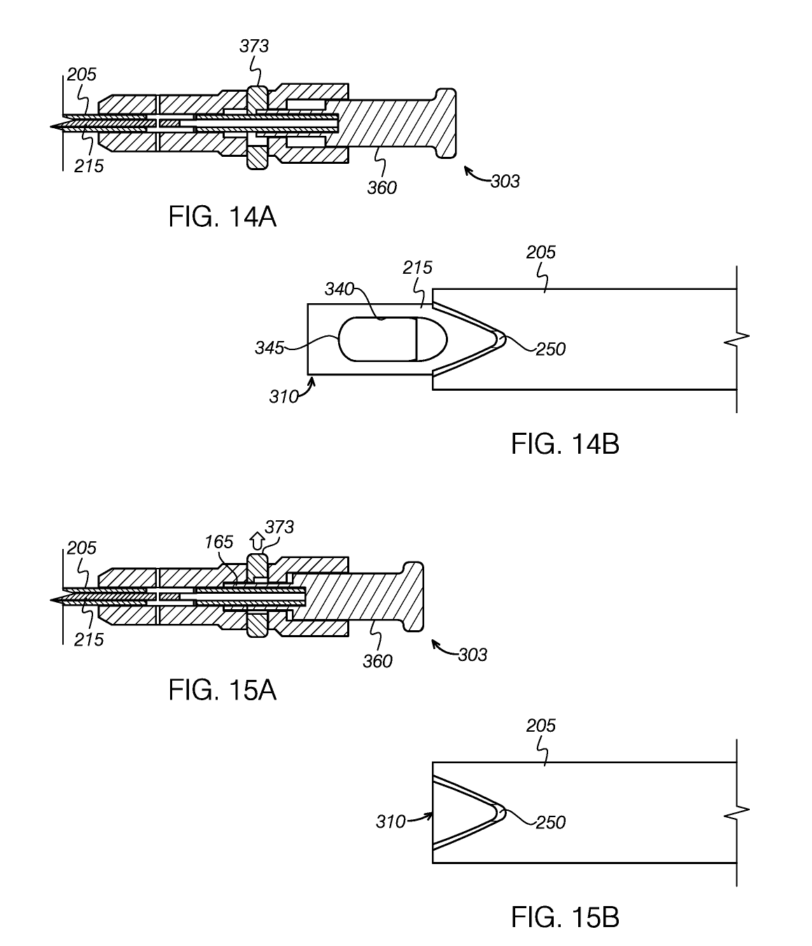

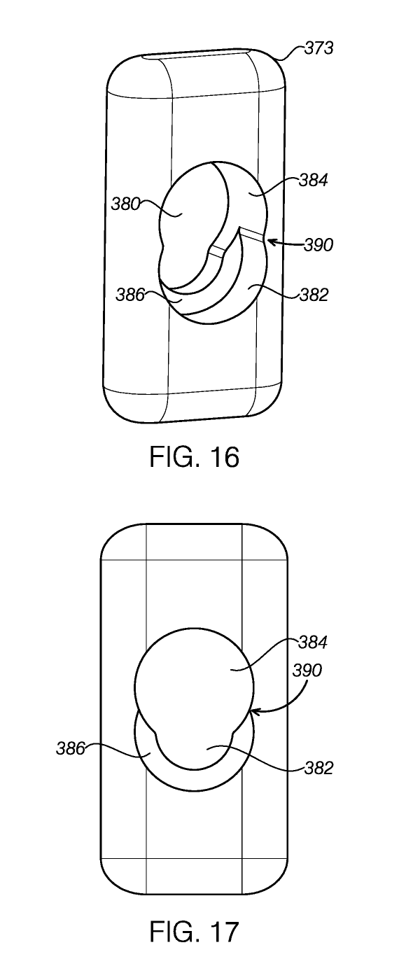

Referring now to FIGS. 14A, 14B, 15A, and 15B show the position of the sleeve 205 relative to the tip 310 for this alternative cutting action with corresponding views of an actuation mechanism 303. The actuation mechanism 303 is similar to the actuation mechanism 103 except that the actuation mechanism 303 includes a gate 373 instead of the gate 173. Referring now to FIGS. 16 and 17 concurrently with FIGS. 14A, 14B, 15A, and 15B, an exemplary embodiment of the gate 373 is shown. The gate 373 may include a slot 380 that has two sections: a locking section 382 and a non-locking section 384. As in other embodiments, the distal end of sleeve 205 may be coupled to a rod 165 that passes through the gate 373. If the rod 165 is in the non-locking section 384, the device can open and close freely by pushing or pulling on the plunger 360. A spring which is not shown can also be placed between the plunger 360 and housing 101 in order to assist motion in one or both directions. If the rod 165 is in the locking section 382 the device has limited travel and can be used to minimize the risk of inadvertent suture cutting. In an exemplary embodiment, a surface of the rod 165 may encounter a locking shoulder wall 386 thus limiting motion. A detent 390 acts as a feature to keep the gate 373 in the locked or unlocked position unless a conscious effort is made to switch positions.

In the cross section of FIGS. 14A and 14B, the device is in the position to load a suture and slide along the suture strand as needed. The gate 373 is positioned such that the rod 165 is restricted in axial movement because of hitting the locking shoulder 386.

In FIGS. 15A and 15B, the gate 373 has been moved to the non-locking position so the suture can be cut. With a distal push of the plunger 360, the sleeve 205 and blade edge 250 move over the blade edge 345 of tunnel exit 340 cutting the suture and covering the tunnel exit 340 in the process.

Although the invention has been discussed with reference to specific embodiments, it is apparent and should be understood that the concept can be otherwise embodied to achieve the advantages discussed. Furthermore, the description is not intended to limit the invention to the form disclosed herein. Accordingly, variants and modifications consistent with the following teachings, skill, and knowledge of the relevant art, are within the scope of the present invention. The embodiments described herein are further intended to explain modes known for practicing the invention disclosed herewith and to enable others skilled in the art to utilize the invention in equivalent, or alternative embodiments and with various modifications considered necessary by the particular application(s) or use(s) of the present invention.

Those of skill in the art would appreciate that various components and blocks may be arranged differently (e.g., arranged in a different order, or partitioned in a different way) all without departing from the scope of the subject technology. For example, a cutting tip shown for one embodiment may be used in conjunction with any of the actuation mechanisms and vice versa. In addition, while the embodiments have been described in terms of the device sleeve being pushed over the cutting tip, alternate embodiments include actuations mechanisms that retract the cutting tip toward the distal edge of the sleeve. In addition, while the embodiments disclosed describe the blade edge being present on both the sleeve and the cutting tip, it will be understood that some embodiments may place the blade on either just the tip or just the sleeve edge or neither the tip or sleeve has a blade but instead opposing walls/wall edges to catch and hold a suture in place. Either the sleeve or the cutting tip may be equipped to position the suture to encounter a blade from the opposing element to be pushed over the tip's blade or the tip's blade retracted onto the sleeve and suture to be cut. The suture can also be threaded through the tip in an opposite direction to that described and cutting still take place.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more.

Terms such as “top,” “bottom,” “front,” “rear,” “above,” “below” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference. Similarly, an item disposed above another item may be located above or below the other item along a vertical, horizontal or diagonal direction; and an item disposed below another item may be located below or above the other item along a vertical, horizontal or diagonal direction. While some features are shown facing away from gravity, for example, the openings in the cutting tips or the blade edges of the sleeves shown, it will be understood that features can be rotated or positioned perpendicular to gravity and work to hold, knot, or cut a suture in the same way as shown.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A suture cutter device, comprising:
a housing;
a handle coupled to the housing;
a shaft coupled to the handle;
a tip of the shaft positioned distally from the handle;
an opening in the tip configured for receiving a suture;
a sleeve surrounding the shaft;
an actuation mechanism for pushing the sleeve over the shaft or retracting the shaft into the sleeve; and
a blade edge positioned on a distal end of the sleeve and disposed to cut the suture in the opening of the tip in response to the actuation mechanism pushing the sleeve over the shaft or retracting the shaft into the sleeve.

2. The suture cutter device of claim 1, wherein the opening in the tip includes a slot and a nook in the slot disposed to position the suture in alignment with the blade edge.

3. The suture cutter device of claim 1, wherein the sleeve includes a tapered guide wall configured to move the suture onto the blade edge.

4. The suture cutter device of claim 1, wherein the opening in the tip includes a hole disposed to position the suture in alignment with the blade edge.

5. The suture cutter device of claim 1, wherein the actuation mechanism is spring-loaded.

6. The suture cutter device of claim 1, wherein the opening in the tip includes an entrance channel defined by a hairpin sidewall including a nook at a terminal point of the hairpin sidewall for securing the suture as the actuation mechanism is operated.

7. The suture cutter device of claim 1, wherein the opening is a pocket hole extending from a first side of the shaft, through a diameter of the shaft, and out a second side of the shaft.

8. The suture cutter device of claim 7, wherein the pocket hole includes a chamfered entrance.

9. The suture cutter device of claim 1, further comprising a second blade edge positioned on the tip of the shaft and in alignment with the blade edge positioned on the distal end of the sleeve.

10. A suture cutter device, comprising:
a housing;
a shaft coupled to the housing;
a tip of the shaft positioned on a distal end of the housing;
a blade edge positioned on an edge of the housing;
an opening in the tip of the shaft configured for receiving a suture; and
an actuation mechanism coupled to the housing for moving the opening in the tip toward the blade edge, wherein the suture in the opening of the tip is cut by the blade edge in response to the actuation mechanism being operated.

11. The suture cutter device of claim 10, further comprising a slot in the opening of the tip and a nook in the slot disposed to position the suture in alignment with the blade edge.

12. The suture cutter device of claim 10, further comprising a tapered guide wall coupled to the housing configured to move the suture onto the blade edge.

13. The suture cutter device of claim 10, wherein the opening in the tip includes a hole disposed to position the suture in alignment with the blade edge.

14. The suture cutter device of claim 10, further comprising an entrance channel defined by a hairpin sidewall including a nook at a terminal point of the hairpin sidewall for securing the suture as the actuation mechanism is operated.

15. The suture cutter device of claim 10, further comprising a sleeve coupled to the housing, the sleeve housing the shaft, wherein the blade edge is positioned on a distal edge of the sleeve in alignment with the suture in the opening in the tip.

16. A suture cutter device, comprising:
a housing;
a handle coupled to the housing;
a shaft coupled to the handle;
a tip of the shaft positioned distally from the handle;
an opening in the tip configured for receiving a suture;
a sleeve surrounding the shaft;
a spring-loaded actuation mechanism for pushing the sleeve distally over the shaft or retracting the shaft into the sleeve;
a first blade edge positioned on a distal end of the sleeve and a second blade edge positioned on the tip of the shaft in alignment with the first blade edge, and the first blade edge and the second blade edge disposed to cut the suture in the opening of the tip in response to the actuation mechanism pushing the sleeve over the shaft or retracting the shaft into the sleeve, wherein the opening in the tip includes a slot and a nook in the slot disposed to position the suture in alignment with the blade edge.

17. The suture cutter device of claim 16, wherein:
the sleeve includes a tapered guide wall configured to move the suture onto the blade edge, and
the opening in the tip includes a hole disposed to position the suture in alignment with the blade edge.

18. The suture cutter device of claim 17, wherein the opening in the tip includes an entrance channel defined by a hairpin sidewall including a nook at a terminal point of the hairpin sidewall for securing the suture as the actuation mechanism is operated.

19. The suture cutter device of claim 16, wherein the opening is a pocket hole extending from a first side of the shaft, through a diameter of the shaft, and out a second side of the shaft.

20. The suture cutter device of claim 19, wherein the pocket hole includes a chamfered entrance.
